(19) United States
(12) Patent Application Publication
(10) Pub. No.: US 2020/0022834 A1
(43) Pub. Date: Jan. 23, 2020

(54) HYGIENE ADHESIVE

(71) Applicants: Susan Elizabeth VanPelt, Kirbyville, TX (US); Jonathan Paul Faro, Houston, TX (US)

(72) Inventors: Susan Elizabeth VanPelt, Kirbyville, TX (US); Jonathan Paul Faro, Houston, TX (US)

(21) Appl. No.: 16/041,240

(22) Filed: Jul. 20, 2018

(51) Int. Cl. A61F 5/455; A61F 13/472

(52) U.S. Cl. CPC A61F 5/455; A61F 13/49; A61F 13/472

(57) ABSTRACT

A hygiene product is provided having a waterproof layer with a perimeter and an adhesive strip disposed around the perimeter of the waterproof layer. The adhesive strip has a body-facing side and an air-facing side, wherein the body-facing side has an adhesive material that is configured to adhere to a user's body and the air-facing side has an adhesive material that is configured to adhere to the perimeter of the waterproof layer. The hygiene product also includes a pull tab attached to the waterproof layer, wherein the pull tab is configured to detach at least a portion of the waterproof layer from the adhesive strip to define an opening through the adhesive strip and the waterproof layer is re-attachable to the adhesive strip after the at least a portion of the waterproof layer is detached from the adhesive strip.

### FIELD OF THE DISCLOSURE

The present disclosure generally relates to a novel disposable feminine hygiene product. More particularly, the present disclosure relates to a disposable adhesive feminine hygiene product used to prevent vaginal irritation.

### BACKGROUND OF THE INVENTION

Many women experience vaginal irritation, and often, the inciting event may be as innocuous as swimming in a public pool. Not only does the irritation that develops lead to a high number of women scheduling appointments to see their physicians (estimates point to more than 10 million visits per year for this reason alone, with over $250 million spent in over-the-counter sales annually), but many women refrain from participating in certain activities out of fear of developing vaginal irritation. Thus, there is a need for a product which prevents women from experiencing vaginal irritation when participating in activities such as swimming. There further exists a need for this product to be easy to use, cheap, and possibly disposable.

### BRIEF SUMMARY OF THE INVENTION

In one aspect of the present disclosure, a hygiene product is provided. The hygiene product comprises a waterproof layer comprising a perimeter. The hygiene product also comprises an adhesive strip disposed around the perimeter of the waterproof layer, the adhesive strip comprising a body-facing side and an air-facing side, wherein the body-facing side comprises an adhesive material that is configured to adhere to a user's body and the air-facing side comprises an adhesive material that is configured to adhere to the perimeter of the waterproof layer. The hygiene product further comprises a pull tab attached to the waterproof layer, wherein the pull tab is configured to detach at least a portion of the waterproof layer from the adhesive strip to define an opening through the adhesive strip, wherein the waterproof layer is re-attachable to the adhesive strip after the at least a portion of the waterproof layer is detached from the adhesive strip.

In another aspect of the present disclosure, the hygiene product may further comprise an absorbent layer, wherein the absorbent layer is attached to the waterproof layer and is disposed on a body-facing side of the hygiene product while the waterproof layer is disposed on an air-facing side of the hygiene product.

In another aspect of the present disclosure, the pull tab may be further attached to the absorbent layer, wherein the pull tab is configured to detach at least a portion of the waterproof layer and the absorbent layer from the adhesive strip to define the opening through the adhesive strip, wherein the waterproof layer and the absorbent layer are re-attachable to the adhesive strip after the at least a portion of the waterproof layer and the absorbent layer is detached from the adhesive strip.

In another still further aspect of the present disclosure, the hygiene adhesive may further comprise a removable strip attached to the body-facing side of the adhesive strip, wherein the removable strip is configured to be detached from the adhesive strip to expose the adhesive material of the adhesive strip. In another aspect, the removable strip may comprise a plurality of removable strips, wherein each of the plurality of removable strips are individually removable from the adhesive strip to expose a corresponding portion of the adhesive material of the adhesive strip.

In yet another aspect of the present disclosure, the adhesive material on the air-facing side of the adhesive strip may extend around only a portion of the perimeter of the waterproof layer.

### BRIEF DESCRIPTION OF THE DRAWINGS

The disclosure will be more readily understood in view of the following description when accompanied by the below figures and wherein like reference numerals represent the elements, wherein:

FIG. 1 illustrates one embodiment of a hygiene product from an air-facing side according to the present disclosure.

FIG. 2 illustrates one embodiment of a hygiene product from a body-facing side.

FIG. 3 illustrates one embodiment of a hygiene product from an air-facing side.

FIG. 4 illustrates one embodiment of a hygiene product applied to a woman's genitals.

### DETAILED DESCRIPTION OF THE INVENTION

The following disclosure as a whole may be best understood by reference to the provided detailed description when read in conjunction with the accompanying drawings, drawing description, abstract, background, field of the disclosure, and associated headings. Identical reference numerals when found on different figures identify the same elements or a functionally equivalent element. The elements listed in the abstract are not referenced but nevertheless refer by association to the elements of the detailed description and associated disclosure.

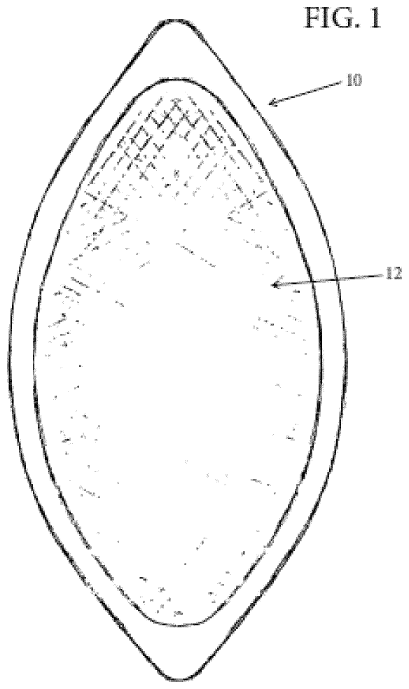

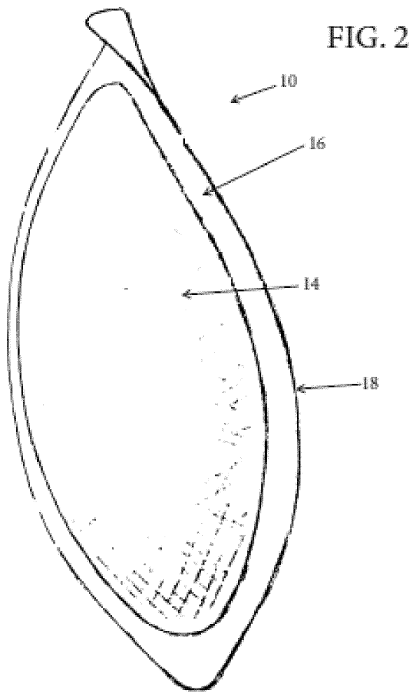

FIGS. 1-3 show one embodiment of a hygiene product 10. FIG. 1 shows an air-facing side of the hygiene product 10 while FIG. 2 shows a body-facing side of the hygiene product 10. When in use, the body-facing side of the hygiene product 10 may be in contact with and facing the user's skin while the air-facing side of the hygiene product 10 may be facing away from the user's skin.

As shown in FIG. 1, the air-facing side of the hygiene product 10 may include a waterproof layer 12. The waterproof layer 12 may be made of a variety of waterproof materials, including but not limited to waterproof polymers such as TEGADERM®.

As shown in FIG. 2, the body-facing side of the hygiene product 10 may include an absorbent layer 14. The absorbent layer 14 may be made of a variety of absorbent and, ideally, non-adherent materials, including but not limited to cotton, rayon, vicose, or a wood fluff pulp-based material. The body-facing side of the hygiene product 10 may also include an adhesive strip 16 that extends along the perimeter of the hygiene product 10. The adhesive strip 16 may be made of a variety of adhesive materials. While this embodiment shows a single adhesive strip 16, the hygiene product 10 may also have multiple adhesive strips. A removable strip 18 (or multiple strips in other embodiments) may also extend along the perimeter of the hygiene product 10 and on top of the adhesive strip 16. Alternatively, the removable strip 18 may extend across the entire hygiene product 10. The removable strip 18 may be used to protect the adhesive strip 16 and prevent it from inadvertently adhering to surfaces before the user intends to use and apply the hygiene product 10. The removable strip 18 may be pulled away from the adhesive strip 16 to expose the adhesive strip 16 to allow the adhesive strip 16 to be applied to the user's body. The removable strip 18 may be made of a variety of materials, including but not limited to an absorbent cotton. In other embodiments, multiple removable strips may be utilized to allow the user to selectively expose and apply portions of the adhesive strip 16 as desired.

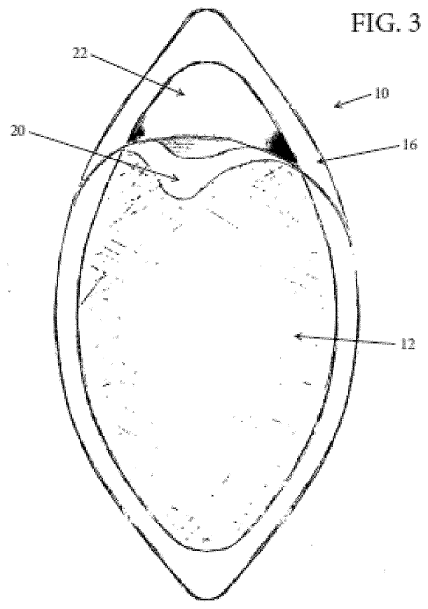

FIG. 3 also shows the air-facing side of the hygiene product 10. The air- facing side of the hygiene product 10 may further include a pull tab 20 for temporarily exposing an opening 22 through the middle of the hygiene product 10. When a user pulls on the pull tab 20, the waterproof layer 12 and absorbent layer 14, which are bonded together via various methods known in the art, may also be pulled with the pull tab 20, thereby partially detaching the waterproof layer 12 and absorbent layer 14 from the adhesive strip 16. When in use, the adhesive strip 16 may remain adhered to the patient's body, thus exposing opening 22 through the middle of the adhesive strip 16. In this embodiment, the adhesive strip 16 has an adhesive material on both the air-facing side and the body-facing side. Thus, when the pull tab 20 is used to pull the waterproof and absorbent layers 12, 14 away from the adhesive strip 16, it may then be reattached to the adhesive strip 16 as desired to the adhesive characteristics of the strip 16. In other embodiments, the adhesive strip 16 may be made of two or more separate strips, including at least one that faces the body-facing side and one that faces the air-facing side. In one embodiment, the air-side of the adhesive strip 16 extends only around a portion of the perimeter of the hygiene product 10. This prevents the user from accidentally removing the waterproof and absorbent layers 12, 14 entirely from the adhesive strip 16.

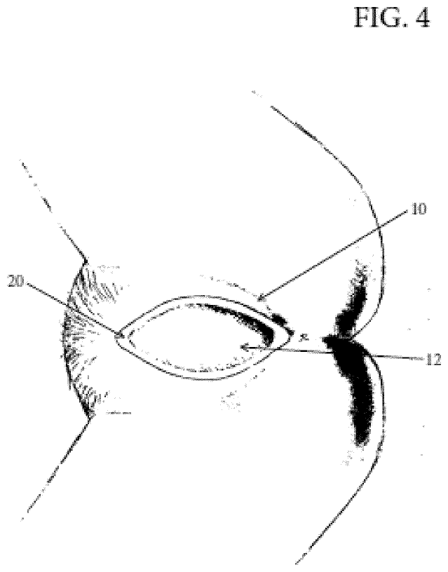

In use and as shown in FIG. 4, the hygiene product 10 may be applied to a woman's genitals for temporary protection from external contaminants when engaged in various activities, such as swimming or bathing. To apply the hygiene product 10, a user may first remove the removable strip 18 (or strips) from the adhesive strip 16 to expose the adhesive strip 16. The hygiene product 10 may then be pressed against and around the user's vagina, with the adhesive strip 16 providing a seal around the user's vagina and the absorbent pad aligned over the user's vagina. When the adhesive strip 16 is properly adhered to the user's skin around the vagina, the waterproof layer 12 provides a watertight or substantially watertight barrier between the user's vagina and the external environment. Thus, the hygiene product 10 will prevent or at least limit the amount of contaminants that the user's genitals are exposed to during activities such as swimming or bathing. Moreover, because the absorbent layer 14 is disposed over the user's vagina, it may help absorb small amounts of bodily fluids, such as menstrual blood or urine, thus keeping the user comfortable throughout use of the hygiene product 10.

If the user needs to urinate during use of the hygiene product 10, the user can pull on the pull tab 20 to separate the waterproof and absorbent layers 12, 14 from the adhesive strip 16, thereby exposing the opening 22 through which the user may urinate. After urination, the user may then reattach the waterproof and absorbent layers 12, 14 to the adhesive strip 16, thereby closing the opening 22 and reestablishing a watertight barrier between the user's vagina and the external environment.

While the present embodiment describes a hygiene product 10 that has an oval shape, the hygiene product 10 may be a variety of shapes and sizes. For example, the hygiene product 10 may be circular or rectangular in shape. Further, the hygiene product 10 may be designed to have multiple sizes to accommodate different users.

While the disclosure herein is focused on the use of the invention to limit contamination of a user's vagina during swimming and other activities, the disclosure is not so limited. Rather, this invention can be used with other body parts that require similar protection from external contaminants, as desired.

The above detailed description and the examples described therein have been presented for the purposes of illustration and description only and not by limitation. It is therefore contemplated that the present disclosure cover any and all modifications, variations or equivalents that fall within the spirit and scope of the basic underlying principles disclosed above and claimed herein.

1. A hygiene product comprising:
a waterproof layer comprising a perimeter;
an adhesive strip disposed around the perimeter of the waterproof layer, the adhesive strip comprising a body-facing side and an air-facing side, wherein the body-facing side comprises an adhesive material that is configured to adhere to a user's body and the air-facing side comprises an adhesive material that is configured to adhere to the perimeter of the waterproof layer; and
a pull tab attached to the waterproof layer, wherein the pull tab is configured to detach at least a portion of the waterproof layer from the adhesive strip to define an opening through the adhesive strip, wherein the waterproof layer is re-attachable to the adhesive strip after the at least a portion of the waterproof layer is detached from the adhesive strip.

2. The device of claim 1, further comprising an absorbent layer, wherein the absorbent layer is attached to the waterproof layer and is disposed on a body-facing side of the hygiene product while the waterproof layer is disposed on an air-facing side of the hygiene product.

3. The device of claim 2, wherein the pull tab is further attached to the absorbent layer, wherein the pull tab is configured to detach at least a portion of the waterproof layer and the absorbent layer from the adhesive strip to define the opening through the adhesive strip, wherein the waterproof layer and the absorbent layer are re-attachable to the adhesive strip after the at least a portion of the waterproof layer and the absorbent layer is detached from the adhesive strip.

4. The device of claim 3, further comprising a removable strip attached to the body- facing side of the adhesive strip, wherein the removable strip is configured to be detached from the adhesive strip to expose the adhesive material of the adhesive strip.

5. The device of claim 4, wherein the removable strip comprises a plurality of removable strips, wherein each of the plurality of removable strips are individually removable from the adhesive strip to expose a corresponding portion of the adhesive material of the adhesive strip.

6. The device of claim 5, wherein the adhesive material on the air-facing side of the adhesive strip extends around only a portion of the perimeter of the waterproof layer.

7. The device of claim 1, wherein the adhesive material on the air-facing side of the adhesive strip extends around only a portion of the perimeter of the waterproof layer.

8. The device of claim 1, further comprising a removable strip attached to the body-facing side of the adhesive strip, wherein the removable strip is configured to be detached from the adhesive strip to expose the adhesive material of the adhesive strip

9. The device of claim 8, wherein the removable strip comprises a plurality of removable strips, wherein each of the plurality of removable strips are individually removable from the adhesive strip to expose a corresponding portion of the adhesive material of the adhesive strip.
